(19) United States
(12) Patent Application Publication
(10) Pub. No.: US 2023/0130056 A1
(43) Pub. Date: Apr. 27, 2023

(54) ANTI-CLENCHING TRAINING DEVICE

(71) Applicant: Bussert Medical, Inc., Nashville, IN (US)

(72) Inventor: Jessica Bussert, Nashville, IN (US)

(73) Assignee: Bussert Medical, Inc, Nashville, IN (US)

(21) Appl. No.: 17/512,056

(22) Filed: Oct. 27, 2021

(51) Int. Cl. A61B 5/00; A61B 5/053

(52) U.S. Cl. CPC A61B 5/4557; A61B 5/0002; A61B 5/053; A61B 5/682; A61B 5/746; A61B 5/6832; A61B 5/7405

(57) ABSTRACT

A muscle anti-clenching training device comprising a flexible device body conformable in a range of configurations upon a user’s body, and including: a skin-contact side and a top cover side; electrical contacts mounted in the flexible device body and exposed upon the skin-contact side; an adhesive upon the skin-contact side; an alert device mounted in the flexible device body; sensing circuitry electrically connected to the electrical contacts and structured to monitor an electrical property in a user’s body; and an electronic controller coupled to the sensing circuitry and structured to activate the alert device to produce an alert signal where the monitored electrical property is indicative of a muscular contraction.

### CROSS REFERENCE TO RELATED APPLICATIONS

The present application claims priority to US Provisional Patent Application No. 63/105,994 filed on Oct. 27, 2020, the disclosure of which is incorporated herein by reference.

### TECHNICAL FIELD

The present disclosure relates generally to an anti-clenching training device.

### BACKGROUND

Those who experience unwanted muscle tensioning and jaw clenching may suffer from negative side effects, such as headaches, jaw pain, tooth pain, or dental problems. Grinding or clenching of the teeth can occur both consciously and subconsciously, which renders such habits difficult to treat. Other related forms of jaw clenching, teeth grinding, and other muscle clenching habits are well-known. For example, bruxism, which is an oral motor condition that results in the grinding or clenching of teeth, is one of the leading causes of temporal mandibular joint dysfunction (TMJ).

Current methods of treating or preventing teeth clenching and/or grinding, including intraoral devices, medications, and contingent electrical stimulation, are expensive, obtrusive, ineffective, and often uncomfortable. For instance, an occlusal splint, which is a large device that is inserted into the mouth and fitted to cover the entire mandibular arch, may be used for those who subconsciously grind their teeth or clench their jaw muscles. These large devices can be dangerous for patients who suffer from sleep apnea and generally fail to produce lasting corrected behavior. Few medications have endured extensive clinical testing, and electrical stimulation tends to be short-term as it may become ineffective after discontinuing the therapy. Some victims of undesired muscle clenching and tensioning conditions may choose a less-intrusive approach to healing by focusing on behavioral changes. Discontinuing bad habits, such as smoking and drinking, are encouraged when seeking therapy but are not proven to be effective in the treatment of bruxism or other, similar muscle clenching conditions.

It is evident that there is a continuing need for treatment, prevention, and generalized therapies in this field. Strategies are needed that focus on correcting and eventually changing learned behaviors, instead of only reducing the effects of these undesirable and unhealthy teeth clenching and muscle tensioning habits.

### SUMMARY OF THE INVENTION

In one aspect, an adhesive, unobtrusive, low-profile flexible training device that alerts a user of muscle clenching, particularly in the jaw or neck muscles, is described. A flexible device body may be multi-layered and may include a flexible printed circuit board with a skin-contact side and a top cover side. The device may be conformable in a range of configurations to the skin of a user’s body. Electrical contacts are positionable against the skin of a user’s body in a range of configurations. The skin-contact side may include a biocompatible adhesive that allows the training device to stick to a user’s skin. Placement over the neck or jaw muscles, for example, allows the device to detect changes in electrical properties which are indicative of muscle contraction and/or clenching. When persistent high voltage levels are detected over a series of voltage scans, a user may be notified through an alert signal, to encourage a user to release the tension within those muscles.

In another aspect, a method for using a low-profile, unobtrusive, flexible training device is provided. Training functions of the device may be initiated once the multi-layer device is placed on a user’s skin and manually turned on by activating an on/off switch. The device may, through a plurality of alternating read and wait cycles, alert a user if voltage scans are consistently at a level that is indicative of muscle contraction. For example, once a user turns the training device on, the device may remain in a passive state while adhered to the user’s skin. After a period of time determined by an internally programmed algorithm elapses, the device initiates an active read cycle. Then, the device may revert back to a passive state and sit passively on a user’s skin until the next active read cycle begins. If the training device detects persistent clenching over multiple cycles, it may send a signal to the alert device to alert a user with a user-perceptible signal.

### BRIEF DESCRIPTION OF THE DRAWINGS

FIG. 1 is a layout view that shows components of a flexible training device according to one embodiment;

FIG. 2 is another layout view that shows components of the flexible training device according to one embodiment;

FIG. 3 is a bottom view of the flexible training device that shows example placement and exposure of electrical contacts according to one embodiment;

FIG. 4 is a block diagram showing relationships between parts of the training device in a flexible printed circuit board according to one embodiment; and

FIG. 5 is an exploded partial cross sectional view of a flexible training device according to one embodiment.

### DETAILED DESCRIPTION

A flexible anti-clenching training device 5 (hereinafter “training device 5”) that uses a flexible printed circuit board 26 is described. A flexible printed circuit board 36, with a circuitry pattern formed thereon, electrically connects internal electronic components that are responsible for controlling the device 5. The flexible printed circuit board may contain a top surface 27 and an oppositely disposed bottom surface 28. Integral components of the device 5 that are distinct from the printed circuitry but function for operating the device 5 may be soldered, clamped, or otherwise attached to a top surface 27 of the printed circuit board 26. The bottom surface 28, eventually to form an exposed outer side of the device, may be coated in a rubberized, grippable material. To form the physical structure of the device 5, the flexible printed circuit board 26 may be folded in half so that the internal electrical connections of the top surface 27 may be made, while the bottom surface 28 of the printed circuit board 26 may be exposed. Folding the printed circuit board 26 in this way also creates two surfaces: a skin-contact side 20 and a top cover side 23. The skin-contact side 20 may be in direct contact with a user’s skin while the oppositely disposed top cover side 23 is accessible to a user. Electrical contact voids 16 may be located on the skin-contact side 20 of the printed circuit board 26 so that electrical contacts 40, which may be mounted to the bottom surface 28 of a printed circuit board 26, may protrude through the electrical contact voids 16 and be in direct contact with a user’s skin. The electrical contact voids 16 may be dispensed with, however, and suitable physical placement of components and electrical connections formed otherwise. A biocompatible adhesive 21, which is further discussed herein, may be present on the skin contact side 20, such as upon the electrical contacts 40.

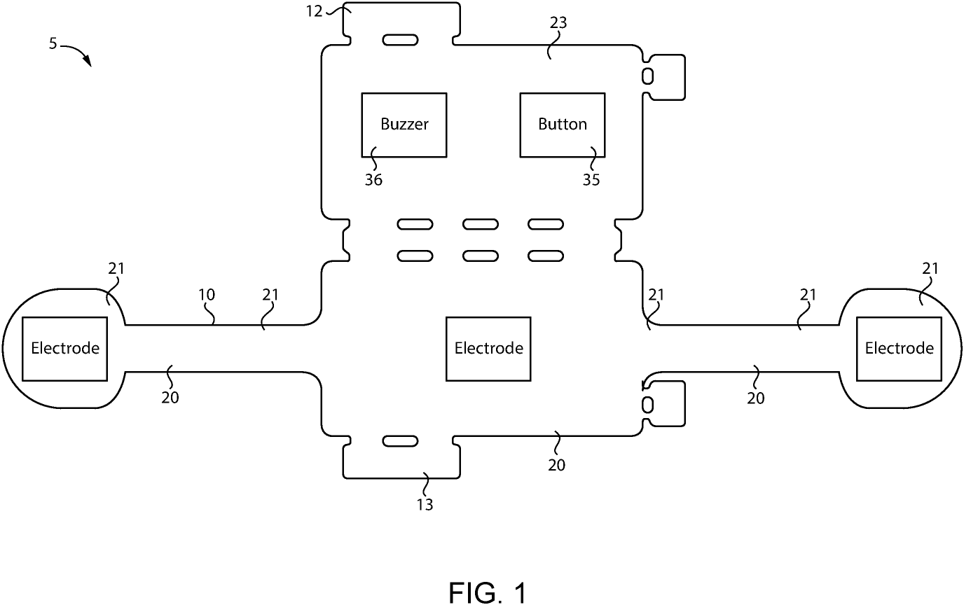

Referring in particular now to FIG. 1, there is shown a training device 5 according to one embodiment. FIG. 1 shows the exposed surfaces of both a top cover side 23 and a skin-contact side 20 of a training device 5 as it might appear in an opened configuration before the device 5 is folded in half. An on/off switch 35 and a speaker/buzzer 36 may be located on a top cover side 23 of the device 5 and may be accessible to a user. As mentioned previously, the top cover side 23 may be made of or include a flexible, rubberized fabric material or other materials, including plastic, cotton, foam, or materials available under the tradename Tricot™. The skin-contact side 20 may be covered in a biocompatible adhesive 21 which allows the training device 5 to stick to a user’s skin. The adhesive 21 may be made of acrylate or latex and is covered with a peel-away backing 22 that is to be removed before placement on a user’s skin. The adhesive 21 may be electrically conductive.

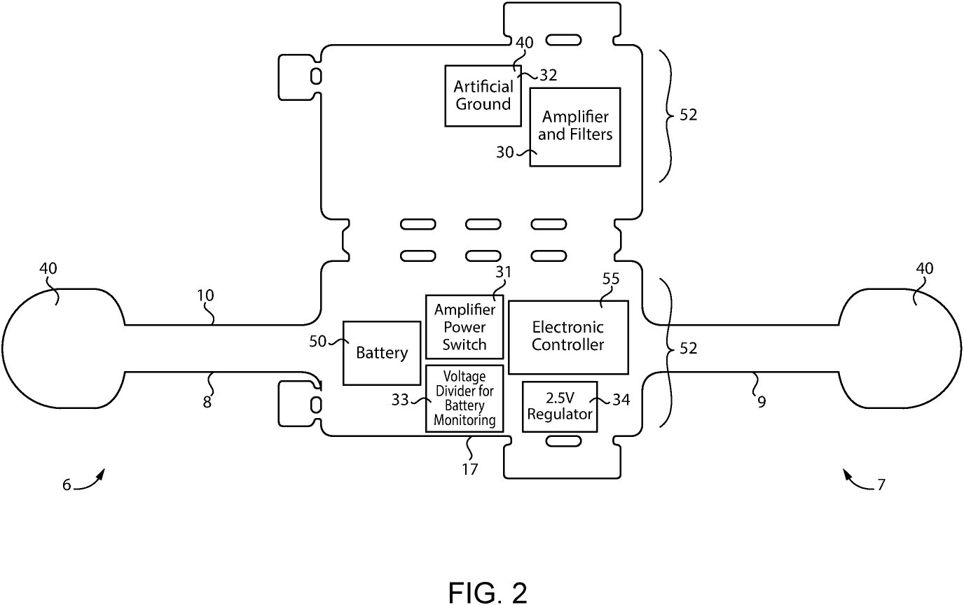

Referring to FIG. 2, there is shown an internal view of electrical components of a training device 5. A printed circuit board 26 creates a flexible device body 10 that may be elongate with a first body end 6 and a second body end 7. Within or forming the first body end 6 is a first elongate strip 8, and within or forming the second body end 7 is a second elongate strip 9. A middle section 17 of the device body 10 may be enlarged laterally, relative to the first elongate strip 8 and the second elongate strip 9, and may contain an amplifier power switch 31, a battery 50, a voltage divider 33, a 2.5 V regulator 34, and an electronic controller 55, such as a microprocessor, microcontroller, or other central processing unit (CPU). The electrical contacts 40 may be distributed longitudinally along the device body 10. One of the three electrical contacts 40 may act as an artificial ground 32 to provide DC voltage to the amplifier 30. This allows the amplifier 30 to produce a signal that may be interpreted by the electronic controller 55.

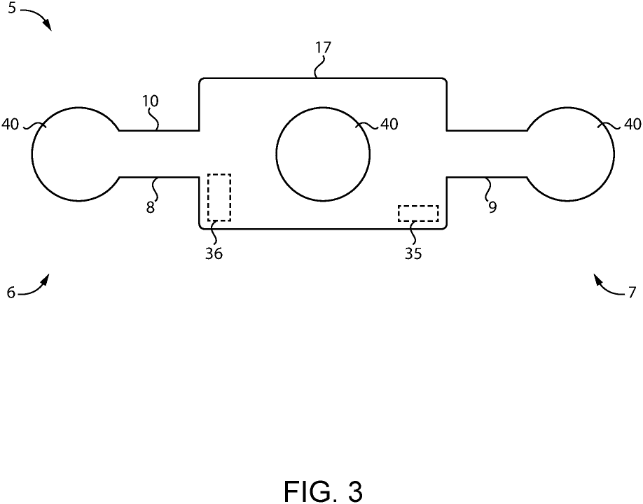

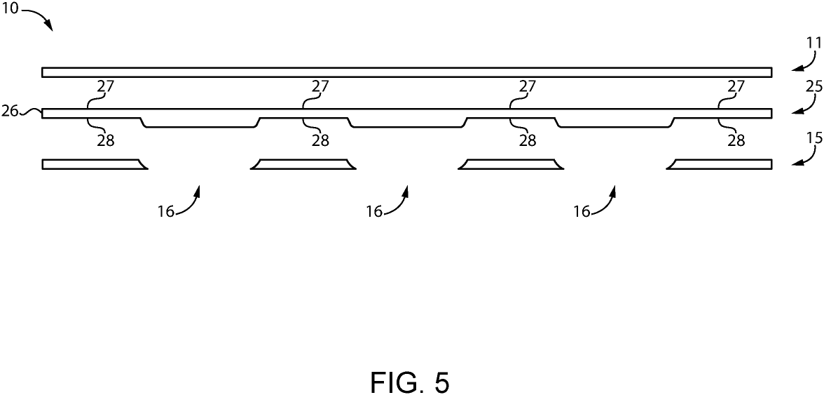

Referring to FIG. 3, there is shown a bottom view of the training device 5 with example placement of electrical contacts 40 within the device body 10. The dotted lines show example relative placement of the on/off switch 35 and the speaker/buzzer 36 on a top cover side 23. A first electrical contact 40 may reside in the first elongate strip 8 within the first body end 6, a second electrical contact 40 may reside in the second elongate strip 9 of the second body end 7, and a third electrical contact 40 may reside in the middle section 17 of the training device 5. A biocompatible adhesive 21 may be upon the electrical contacts 40 and may be made of electrically conductive materials, such as conductive silicon rubber or another suitable material. The electrical contacts 40 may be placed directly on top of and extend across the muscle(s) intended to be monitored and may be in constant contact with a user’s skin. For example, one of the three electrical contacts 40 may be intentionally placed over the head of a muscle, a second electrical contact 40 may be placed medially over a muscle, and a third electrical contact 40, which may be identified as the artificial ground 32, may be intentionally placed away from either of those previously mentioned areas. As previously mentioned, electrical contact voids 16 may be located on the skin-contact side 20 of the printed circuit board 26 so that electrical contacts 40, which may be mounted to a bottom surface 28 of the printed circuit board 26, may protrude through the electrical contact voids 16 and be in direct contact with a user’s skin. FIG. 5 shows an exploded, partial cross sectional view of a training device 5. In this particular view the top cover portion 11, middle/internal portion 25, and skin contact/bottom portion 15 are visible.

Generally, electrical signals are emitted when jaw or neck muscles are clenched or tightened. These signals may be detected by the electrical contacts 40, transmitted through the amplifier 30, picked up by the electronic controller 55, which may be coupled to the sensing circuitry 52, and translated to activate the wireless alert device 45. The wireless alert device 45 can be either a speaker or buzzer 36 capable of generating audio and/or tactile alerts. For example, in some embodiments, a Piezoelectric speaker may be used as the wireless alert device. The wireless alert device 45 may create an audible signal if the electrical contacts 40 detect a constant or repetitive increase in voltage that is indicative of muscle contraction over a series of alternating active and passive cycles. The alert signal 46 may then be triggered to notify a user of their clenched or tense muscles. The user-perceptible signal may be communicated wirelessly through other methods, including an application on a cellular device, or an earpiece.

In some embodiments, a sensor assembly 60 may include the sensing circuitry 52, the electronic controller 55, and electrical contacts 40. Further, the sensor assembly 60 may also include a rechargeable and/or replaceable power supply that provides power to the device 5. Multiple variations of a power supply may be used, but a practical strategy uses a battery 50. More specifically, an embodiment may use a single-use battery, a rechargeable battery, or a plug-in AC/DC power supply. Other embodiments include a Universal Services Bus (USB) port for connecting a sensor assembly 60 to a power supply and/or data communication link. The power supply may be controlled by an on/off switch 35 that may be situated in the middle section 17 and may be exposed on a top cover side 23. The on/off switch 35 may be readily accessible to and controlled by a user. In still other embodiments the sensor assembly may include components which allow the assembly to communicate data and/or charge wirelessly such as IEEE 802.11 standards (WiFi), IEEE 802.15 (Bluetooth®), or other suitable standards.

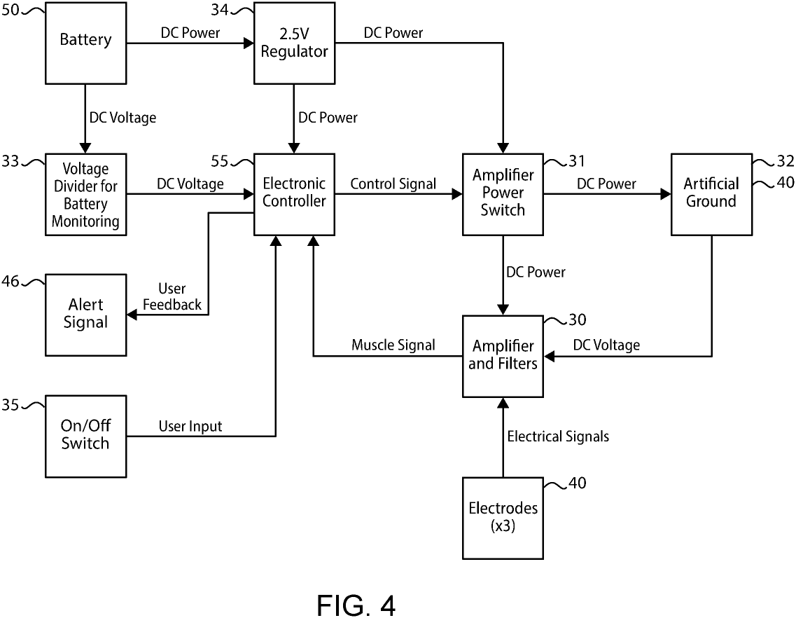

Turning now to FIG. 4, there is shown a block diagram of the electrically communicative relationships between parts of the training device 5 that may be located inside or upon the flexible printed circuit board 26. Activity sensed by the electrical contacts 40 may be transferred through the amplifier 30 and to the electronic controller 55. The electronic controller 55 may then signal the wireless alert device 45 to produce a user-perceptible alert signal 46 which notifies a user of their actions.

In one embodiment, activation of the on/off switch 35 may place the device 5 in a passive state, meaning the device is powered on but may not be actively measuring the electrical output of a particular muscle at that time. Once the allotted time for a passive state elapses, the device 5 may switch to an active state and simultaneously initiate a voltage reading. After the electrical output of a muscle is measured and recorded, the device 5 may revert to a passive state. An internal algorithm controls the length of time between each active and passive state. These cycles only occur when the on/off switch 35 is in the “on” position. If consistent voltage readings that are indicative of muscle clenching or tensioning are identified, the device may trigger the wireless alert device 45 to produce an alert signal 46 intended to notify a user of their actions. In other embodiments, the device 5 in the “on” state may actively record and measure voltage readings in a particular muscle until the device 5 is physically switched to the “off” position. Regardless of whether monitoring is continuous, intermittent, or takes still another form, a user can be trained by way of the feedback as to their muscle tensioning or clenching behavior to reduce or eliminate the behavior over time. The detection strategy may be less sensitive in the beginning to detect relatively hard or frequent muscle clenching only, and may become more sensitive over time. This type of strategic variation in sensitivity may help train a user to recognize and self-regulate their undesirable clenching behavior more consistently over time and avoid unduly frequent or irritating repetition of alerts, enabling the training to more gradually reduce undesired behavior.

The present description is for illustrative purposes only and should not be construed to narrow the breadth of the present disclosure in any way. Thus, those skilled in the art will appreciate that nigh-infinite modifications might be made to the presently disclosed embodiments without departing from the full and fair scope and spirit of the present disclosure. Other aspects, features, and advantages may be apparent upon an examination of the attached drawings and appended claims. As used herein, the articles “a” and “an” are intended to include on or more items, and may be used interchangeable with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

1. A muscle anti-clenching training device comprising:
a flexible device body conformable in a range of configurations upon a user’s body, and including a skin-contact side and a top cover side;
electrical contacts mounted in the flexible device body and exposed upon the skin-contact side;
an adhesive upon the skin-contact side;
an alert device mounted in the flexible device body;
sensing circuitry electrically connected to the electrical contacts and structured to monitor an electrical property in a user’s body; and
an electronic controller coupled to the sensing circuitry and structured to activate the alert device to produce an alert signal where the monitored electrical property is indicative of an undesired muscular contraction.

2. The training device of claim 1 wherein the alert device includes a wireless alert device, and the alert signal includes a wireless alert signal.

3. The training device of claim 2 wherein the alert device includes an audio alert device and the alert signal includes a user-perceptible audio signal.

4. The training device of claim 3 wherein the alert device includes a piezoelectric transducer.

5. The training device of claim 1 wherein the flexible device body is elongate and extends between a first body end and a second body end, and the electrical contacts are distributed longitudinally along the device body.

6. The training device of claim 5 wherein the first body end includes a first elongate strip and the second body end includes a second elongate strip, and a middle section of the device body is between the first elongate strip and the second elongate strip and is enlarged laterally relative to the first elongate strip and the second elongate strip.

7. The training device of claim 6 wherein a first one of the electrical contacts is mounted in the first elongate strip, a second one of the electrical contacts is mounted in the second elongate strip, and a third one of the electrical contacts is mounted in the middle section.

8. The training device of claim 6 wherein the sensing circuitry, the alert device, and the electronic controller are all resident in the middle section.

9. The training device of claim 5 wherein a number of the electrical contacts is three.

10. The training device of claim 9 wherein the adhesive is upon the electrical contacts.

11. The training device of claim 1 wherein the flexible device body is formed at least in part by a flexible printed circuit board.

12. The training device of claim 11 wherein the flexible printed circuit board is folded to form a base layer and a second layer, and a cavity is formed between the base layer and the second layer, and the electrical contacts are attached to the base layer.

13. The training device of claim 9 wherein the alert device is mounted upon the second layer.

14. The training device of claim 13 further comprising an ON/OFF switch mounted upon the second layer.

15. The training device of claim 12 further comprising a battery electrically connected to the sensing circuitry and to the electronic controller and positioned in the cavity.

16. The training device of claim 1 wherein the monitored electrical property includes a voltage difference between skin locations on the user’s body corresponding to an undesirable behavior.

17. The training device of claim 16 wherein the sensing circuitry is further structured to monitor the electrical property in a plurality of read cycles alternating with a plurality of wait cycles.

18. The training device of claim 17 wherein the electronic controller is further structured to activate the alert device where the monitored electrical property indicates a muscular contraction in a plurality of the read cycles.

19. The training device of claim 16 wherein the device decreases in sensitivity over time to curb the undesirable behavior.

20. A method of preparing a muscle anti-clenching training device for service in training a user comprising:
coupling an electronic controller to muscle contraction sensing circuitry in a flexible device body of a muscle anti-clenching training device, where the flexible device body includes a skin-contact side and a top side, and electrical contacts exposed upon the skin-contact side;
coupling the electronic controller to an alert device mounted to the flexible device body;
establishing communications between the electronic controller and each of the sensing circuitry and the alert device based on the coupling of the electronic controller to the sensing circuitry and to the alert device, such that the electronic controller activates the alert device to produce an alert signal where a monitored electrical property in a user’s body is indicative of undesired muscle contraction.
